(19) United States
(12) Patent Application Publication
(10) Pub. No.: US 2022/0125964 A1
(43) Pub. Date: Apr. 28, 2022

(54) FIBER OPTIC STERILIZATION DEVICE

(71) Applicant: Hai Solutions, Inc., Carlsbad, CA (US)

(72) Inventors: Paul Swaney, Carlsbad, CA (US); Nicholas Perrenoud, Carlsbad, CA (US); Adnan Abbas, Carlsbad, CA (US)

(21) Appl. No.: 17/429,916

(22) PCT Filed: Feb. 11, 2020
(86) PCT No.: PCT/US2020/017771

§ 371 Date: Aug. 10, 2021

Related U.S. Application Data
(60) Provisional application No. 62/804,044, filed on Feb. 11, 2019.

(51) Int. Cl. A61L 2/00; A61L 2/28; A61C 19/06

(52) U.S. Cl. CPC A61L 2/0052; A61C 17/00; A61C 19/063; A61L 2/28

(57) ABSTRACT

Described herein generally are sterilization devices that can allow sterilization of a subgingival area, periodontal pocket(s), and/or gum tissue. Methods of using these devices are also described.

### CROSS REFERENCE TO RELATED APPLICATION

This application claims the benefit of U.S. Provisional Patent Application No. 62/804,044, filed Feb. 11, 2019, the entire disclosure which is incorporated herein by reference.

### FIELD

The present invention relates generally to sterilization devices.

### SUMMARY

Described herein generally are sterilization devices that can allow a dentist, dental hygienist, dental assistant, physician, nurse, assistant, caregiver, provider, or the like to disinfect or decontaminate a subgingival area, periodontal pocket(s), or gum tissue during normal and routine dental hygiene examinations. Also, described herein are the methods for using the sterilization devices.

The described sterilization devices can include a body and an adapter cap. In some embodiments, the adapter cap is a fiber optic adapter cap. In other embodiments, the fiber optic adapter cap is an optical probe. The optical probe can have a curved tip, a right angle tip, a tip configured to allow light to exist in a wide array, and/or a tip configured to allow light to exist in a narrow circumference. In some embodiments, the fiber optic adapter cap comprises a fiber optic cable(s) and/or filament(s). In other embodiments, the fiber optic cable(s) and/or filament(s) can carry light from the device to site of treatment. In some embodiments, the length of the optic probe can be or short or it can be long ranging from anywhere in between about 0.01 mm to about 5 feet.

In some embodiments, the body can include an activation or on/off switch, at least one indicator light, at least one battery, and a light source. In other embodiments, the body can optionally include a printed circuit board including a processor and memory.

In some embodiments, the body can include an activation or on/off switch, at least one indicator light, a power source, and a light source.

In yet other embodiments, the devices described herein can have independent drivers that do not require a processor. In some embodiments, the independent drivers are LED drivers. In some embodiments, the adapter cap can be a fiber optic adapter cap.

In some embodiments, the fiber optic adapter cap can be biocompatible. In other embodiments the fiber optic adapter cap can be disposable. In some embodiments, the fiber optic adapter cap can be a male luer lock. In other embodiments, the fiber optic adapter cap can be a female luer lock. The luer lock can be a spinning luer lock that freely spins independently of the body. In other embodiments, the luer lock can also be a fixed luer lock that does not spin.

In some embodiments, the devices can be small, lightweight, and/or handheld. However, the devices need not be small, lightweight, and/or handheld and can include a body that is virtually any size or weight.

In some embodiments, the devices can deliver ultraviolet light to sterilize a subgingival area, periodontal pocket(s), or gum tissue. In some embodiments, the ultraviolet light can be ultraviolet C (UVC) light. In other embodiments, the ultraviolet light can be ultraviolet B light.

In one embodiment, the devices can deliver UVC light from a 3.5 mm×3.5 mm light-emitting diode (LED) with a 275 nm wavelength fora 5-10 second interval.

The devices described can effectively denature any microbe or cellular structure on any subgingival area, periodontal pocket(s), or gum tissue.

In some embodiments, the devices can sterilize a subgingival area, periodontal pocket(s), or gum tissue within or in less than about 5 seconds. In other embodiments, the devices can sterilize a subgingival area, periodontal pocket(s), or gum tissue within or in less than about 10 seconds.

In some embodiments, the optical power from the LED can be about 10 mW. In other embodiments, the optical power from the LED can be up to 50 mW.

In other embodiments, the device can use only about 6 volts of power.

In other embodiments, the devices can have a switch that activates the device to start sterilizing. In some embodiments, the devices can have an auto mode wherein the device begins to sterilize as soon as a connection is completed. In some embodiments, the device can have a multi-sensor. In other embodiments, the device can have at least one or more sensors.

In other embodiments, the device can have one or more indicator light(s) that can turn green once sterilization is complete. In some embodiments, the device can have one or more indicator light(s) which are blue and turn green once sterilization is complete. In other embodiments, the indicator light(s) can be, but are not limited to, red, blue, purple, yellow, no color, or orange, which then turn green upon completion of sterilization. In some embodiments, there can be a chromatic scheme. In other embodiments, the indicator light(s) are part of the fiber optic adapter cap.

In other embodiments, the device can have a tactile response to indicate sterilization is complete. In some embodiments, the tactile response is vibration.

In some embodiments, the devices can sterilize the intended space/pocket(s) at a distance of about 1 mm to about 10 mm from the intended space/pocket(s). In some embodiments, the distance is less than about 10 mm. In other embodiments, the distance is greater than about 1 mm.

Methods are also described for using the herein described devices. Methods can include probing subgingival area, periodontal pocket(s), or gum tissue to sterilize the space/pocket(s) within about 5 seconds.

In other embodiments, the methods can include connecting the devices to several fiber optic fibers and sterilizing sequentially one at a time or at the same time. Such sterilization for 1 to 20 ports fibers can take about 30 to about 40 seconds. In other embodiments, methods can include sterilization of 1 to 50 fibers.

In other embodiments, the devices described herein can include an inter-locking circuit. In some embodiments, the inter-locking circuit can be connected to the fiber optic adaptor cap or to a luer to turn on the safety feature. In other embodiments, the inter-locking circuit can have an RFID chip or optical sensor configured to allow the fiber optic adaptor cap and device to communicate. In some embodiments, the fiber optic adaptor cap and device can communicate via physical, mechanical, or optical communication.

### BRIEF DESCRIPTION OF THE DRAWINGS

FIG. 1 illustrates an example embodiment of a fiber optic probe as described herein.

FIG. 2 illustrates a top end view of a fiber optic probe as described herein.

FIG. 3 illustrates a back end view of a fiber optic probe as described herein

FIG. 4 illustrates a transparent view of a fiber optic probe as described herein.

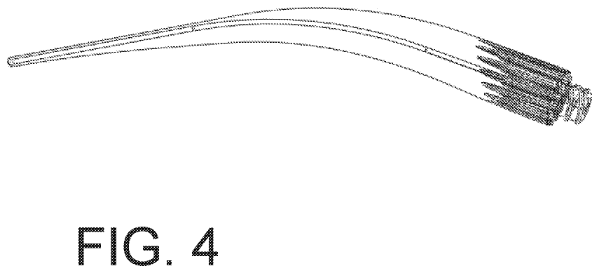

### DETAILED DESCRIPTION

Described herein generally are devices that can reduce, eliminate, and/or prevent chances of infection.

In some embodiments, the devices are sterilization devices utilized for sterilization of a subgingival area, gum tissue, or periodontal pocket(s). Subgingival can include a pocket, a pocket below the gum line, a space below the gum line, or an area below the gum line. In some embodiments, the devices described here can be applied directly onto the tooth/teeth.

In other embodiments, the devices are fiber optic probes utilized for sensing or measuring gum disease and bacterial colonization.

Sterilization of the surfaces and spaces of subgingival area, gum tissue, or periodontal pocket(s) can be very time consuming. In some instances, a patient may require 1 to 20 treatments per month. Sterilization using the described devices can reduce this time burden, in some cases by about 66%.

The primary reason for tooth loss is gum disease where bacteria form a layer of microfilm in and around the gum layer. This bacterial ingress below the gum line can present a chronic and active infection in the subgingival space. Periodontal disease is a set of inflammatory conditions affecting the tissues surrounding the teeth. Early on it's also known as gingivitis, where the gums can become swollen, red, and could bleed. As the disease progresses and inflammation continues the gums can pull away from the teeth and potentially cause bone loss.

Periodontal disease is generally caused by a buildup of bacteria in the mouth infecting the gum tissue around the teeth. Some risk factors include smoking, diabetes, HIV, AIDS, family history, cardiovascular, and certain medications. Diagnosis is made by examining the gum tissue around the teeth both visually and with dental instrumentation. Treatment includes oral hygiene, regular professional teeth cleaning and laser decontamination treatment. In certain cases antibiotics in the form of subgingival antibiotic microspheres are delivered to the site of infection or dosed prophylactically. Dental surgery may be recommended.

Internationally more than 538 million people were affected in 2015. In the US nearly half of those over the age of 30 are affected to some degree, while about 70% of those over the age of 65 have the condition. Males are affected more often than females.

In some embodiments, the devices are sterilization devices utilized for sterilization of endoscopes, working channels, luminal surfaces or borescopes.

In other embodiments, the sterilization devices described herein can comprise a body comprising at least one indicator light, a power source, and at least one light source; and an adapter cap. In some embodiments, the adapter cap can include a fiber optic cable or filament.

Endoscopes are instruments which can be inserted in the human body allowing the physician to gain visualization via a camera system. Endoscopes also contain working channels allowing the physician to advance working instruments required to deliver treatment during a medical procedure. Endoscopes are utilized during a variety of procedures including ENT, General and Gastroenterology procedures. During these procedures working channels can accumulate contaminates like patient debris, tissue and bodily fluids like blood. If not properly decontaminated, endoscopes can also transfer or transmit bacteria, microbes or other types of contamination. Serious or life threatening infections and or death can result due to a lack of decontamination or sterilization.

In some embodiments the fiber optic adapter could utilize a longer disposable extension to deliver UVC light throughout the working channel(s) thereby irradiating, and sterilization parts or sections of the endoscopes. IN other embodiments, the fiber optic adapter could be placed down the channel of a fiber optic endoscope. Fiber optic endoscopes or endoscopes can have three to four tiny ports. In some embodiment, a fiber optic endoscope or endoscope can have a closed fiber optic port for video, a closed fiber optic port for light, and two or more open channels for fluid or section. In some embodiments, the fiber optic adapter can be used for the open channels.

The present devices can sterilize a subgingival area, periodontal pocket(s), gum tissue, or luminal surfaces quicker and more efficiently than even the present standards outline. The present devices can provide clinical benefits that enhance provider efficiency, and improve clinical outcomes while delivering significant cost savings. In some embodiments, the present devices can be built on a device/disposable platform that can deliver complete sterilization and at least a 4-log (99.99%) microbial reduction. This 4-log (99.99%) microbial reduction is greater than standard methods which only promise to deliver disinfection. In other embodiments the microbial reduction can be about a 4-log microbial reduction, about a 5-log microbial reduction, about a 6-log microbial reduction, about a 7-log microbial reduction, about a 8-log microbial reduction, at least about a 4-log microbial reduction, at least about a 5-log microbial reduction, at least about a 6-log microbial reduction, at least about a 7-log microbial reduction, at least about a 8-log microbial reduction, more than a 4-log microbial reduction, more than a 5-log microbial reduction, more than a 6-log microbial reduction, more than a 7-log microbial reduction, more than a 8-log microbial reduction, or between about a 4-log microbial reduction to about a 8-log microbial reduction.

The devices described herein can reduce methicillin-resistant Staphylococcus aureus (MSRA) bacteria on surface(s) or space(s) of a subgingival area, periodontal pocket(s), or gum tissue with 99.99% kill after 1 second, 99.999% kill after 3 seconds, and >99.99999% after 7 seconds of UV light exposure. These results demonstrate the effectiveness of the devices described herein. In comparison, current methods on the market take days to reach only a 98% kill, whereas the devices described herein take a second to achieve a 99.99% kill.

In some embodiments, the devices described herein can be used to sterilize surfaces, spaces, orifices, or cavities in patients. A patient as used herein can be a mammal such as a humans, horses, camels, dogs, cats, cows, bears, rodents, oxen, bison, buffalo, caribou, moose, deer, elk, sheep, goats, pigs, rabbits, pouched mammals, primates, carnivores, and the like. When used with/for patients, the devices can meet the highest safety standards imposed by local, regional, or governmental regulations for sterilization protocols.

According to the Centers for Disease Control and Prevention and American Academy of Periodontology, moderate and severe periodontitis is estimated at 30 percent among adults. The American Dental Association recommends deep cleaning of teeth, scaling, root planning and treatment for chronic periodontitis which can lead to gum disease and tooth loss. Guidelines based on systemic review and treatment of periodontitis are published in the Journal of American Dental Association.

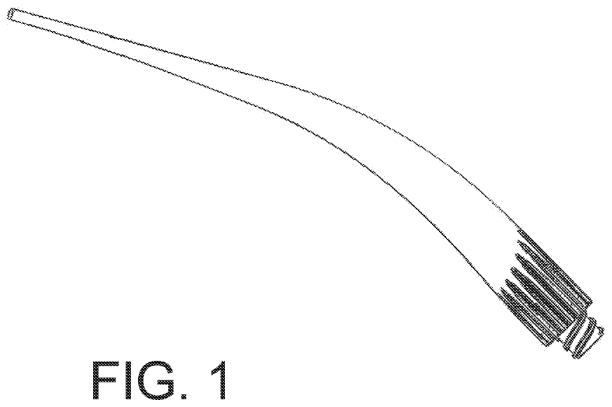

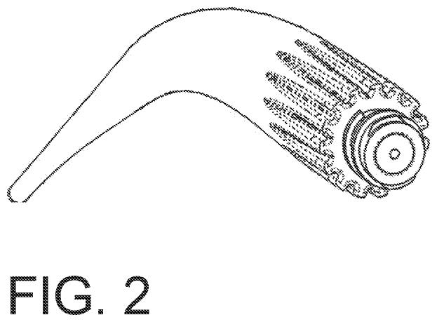

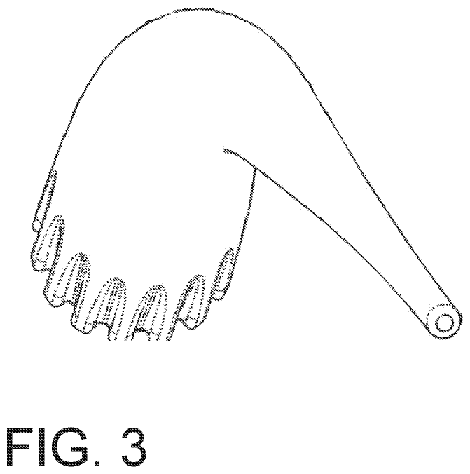

One embodiment of a device as described herein is illustrated in FIGS. 1-3. A device described herein can include a body.

The body can be formed of non-conductive materials such as polymers. Exemplary polymers include, but are not limited to polyurethanes, silicones, polyesters such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; carboxymethyl cellulose; synthetic and natural rubbers such as polysiloxanes, latex, polymerized isoprene, bromo isobutylene isoprene, chloro isobutylene isoprene, polychloroprene, chlorosulphonated polyethylene, ethylene propylene, ethylene propylene diene monomer, fluoro silicone, hydrogenated nitrile butadiene, polyisoprene, isobutylene isoprene butyl, methyl vinyl silicone, acrylonitrile butadiene, acrylonitrile butadiene carboxy monomer, styrene butadiene, epichlorodydrin; and combinations thereof.

In some embodiments, the body can be formed of cyclic olefin copolymers. In other embodiments, the body can be formed of TOPAS®. TOPAS can be used to allow for at least 80% of the UV light to penetrate through it. Many other plastics/polymers will not allow for at least 80% of the UV light to penetrate through it.

The polymer or combination of polymers chosen to form the body can be rigid enough to hold a particular configuration and perform its intended function. In some embodiments, the polymer used is a thermal set rigid plastic. In other embodiments, the polymer is a flexible nylon or rubber polymer.

The body can include or be connected to a fiber optic adapter cap.

In some embodiments, the fiber optic adapter cap can be sterile. In other embodiments, the fiber optic adapter cap can be biocompatible. In some embodiments, the fiber optic adapter cap can be disposable.

In some embodiments, the fiber optic adapter cap can include a fiber optic extension or fiber(s). In other embodiments, the length of the fiber optic extension is 1-2 inch(es), 1 inch, 2 inches, about 1 inch, about 2 inches, at least 1 inch, at least two inches, 1 foot, 2 feet, 3 feet, 4 feet, 5 feet, 6 feet, or any length needed to deliver targeted therapy.

In some embodiments, the fiber optic adapter cap allows for UVC light to be coherent. In other embodiments the fiber optic adapter cap can collimate the light in such a manner as to allow it to be distributed, delivered or magnified to the tip or end of the fiber optic fiber at maximum power.

In some embodiments the fiber optic adapter cap can be rigid, malleable or flexible. In other embodiments the fiber optic adapter cap can allow the user to control the orientation, shape or direction of UV light.

In some embodiments, the fiber optic adapter cap can be threaded to accept a threaded luminal connection port. In other embodiments, the fiber optic adapter cap may not be threaded so that it can be friction fitted to a luminal connection port. In some embodiments, the fiber optic adapter cap can magnetically fit with the luminal connection port. In other embodiments, the device can include a circular recess so that any type of adapter cap or fiber optic adapter cap can fit in it.

In some embodiments, the fiber optic adapter cap can include a silica lens to allow ultraviolet light to be delivered. In some embodiments, the fiber optic adapter cap can include a sapphire lens to allow ultraviolet light to be delivered.

The body can include an activation or on/off switch. The activation or on/off switch can be a push button, a press button, or a toggle button/switch.

In other embodiments, the body may not include a switch. In such an embodiment, the body can include an auto mode wherein the device begins to sterilize as soon as a connection is completed. In some embodiments, the connection can be when the device senses gum tissue. In some embodiments, the device can have at least one or more sensors. In other embodiments, the device can have multi-sensors. In some embodiments, the at least one or more sensors can be located anywhere on the device. In other embodiments, the multi-sensors can be located anywhere on the device.

The devices described herein can include at least one indicator light. The body can include six indicator lights. The indicator light(s) can be one color prior to connecting the fiber optic adapter cap.

Indicator lights on the body of the device can progressively illuminate, for example the light adjacent to the switch or to the light adjacent to the fiber optic adapter cap. This progression can display the sterilization process time.

As described, different light color combinations can be used to indicate device status. In some embodiments, red light can indicate sterilization has not occurred, and green light can indicate proper sterilization. In some embodiments, a blue light can be used to indicate in process. In some embodiments, only a single light is needed to provide status. In other embodiments, multiple lights can be used to provide status.

In some embodiments, there can be a chromatic scheme. In other embodiments, indicator lights can be part of the fiber optic adapter cap. In other embodiments, indicator lights can be located anywhere on the device. In some embodiments, the chromatic scheme can be used to determine the status of the sterilization. In other embodiments, the chromatic scheme can include blue light(s) which indicate sterilization has not occurred. In some embodiments, the chromatic scheme can include green light(s) which indicate proper sterilization. In other embodiments, the chromatic scheme can include red light(s) or orange light(s) which indicate sterilization has not occurred. In some embodiments, the chromatic scheme can include blue light(s) which indicate that sterilization has occurred.

In some embodiments, there is a substrate or liquid situated in a base of the fiber optic adapter cap which can change colors at specific wavelengths. In other embodiments, the substrate or liquid can be clear and turn green upon completion of sterilization. In some embodiments, when the fiber optic adapter cap is exposed to UV light the substrate will change. Once the substrate changes color it can be irreversible so that the fiber optic adapter cap is single use and cannot be used again.

The body can house a power source. In some embodiments, the body can house at least one power source. The power source can be a battery, a plug, a plug connected to or plugged into a wall, or a combination thereof.

The body can house at least one battery. The body can optionally include a printed circuit board including at least one processor and memory. The at least one battery can be any standard sized battery. Standard size batteries can include, but are not limited to round, cylindrical batteries such as AA, AAA, AAAA, C, D, and button cell (such as lithium button), coin cell, and non-round batteries such as box batteries, and the like. In still other embodiments, proprietary battery packs can be provided to fit a particular slot or opening on or in a device. In some embodiments, the device uses a battery providing about 1 volt, about 2 volts, about 3, about 4 volts of power, about 5 volts of power, about 6 volts of power, about 7 volts of power, about 8 volts of power, about 9 volts of power, about 10 volts of power, about 11 volts of power, about 12 volts of power, at least about 4 volts of power, at least about 5 volts of power, at least about 6 volts of power, at least about 7 volts of power, at least about 8 volts of power, at least about 9 volts of power, at least about 10 volts of power, at least about 11 volts of power, at least about 12 volts of power, more than about 4 volts of power, more than about 5 volts of power, more than about 6 volts of power, more than about 7 volts of power, more than about 8 volts of power, more than about 9 volts of power, more than about 10 volts of power, more than about 11 volts of power, more than about 12 volts of power, between about 4 volts of power to about 11 volts of power, or between about 4 volts of power to about 12 volts of power.

In some embodiments, two voltages are provided. Either voltage can be about 9 volts of power, about 9.5 volts of power, about 10 volts of power, about 10.5 volts of power, about 11 volts of power, about 11.5 volts of power, about 12 volts of power, about 12.5 volts of power, about 13 volts of power, about 13.5 volts of power, about 14 volts of power, about 14.5 volts of power, between about 9 volts of power and about 13.5 volts of power, between about 9 volts of power to about 13 volts of power, between about 9 volts of power to about 12 volts of power, or between about 9 volts of power to about 11 volts of power. In other embodiments, a battery can provide two voltages of power, one of about 9 volts of power to about 13.5 volts of power, and another of about 9 volts to about 12 volts.

Battery life can be about 6 months to 1 year. In some embodiments, the life of the battery can be about 28 days, about 30 days, about 1 month, about 2 months, about 3 months, about 4 months, about 5 months, about 6 months, about 7 months, about 8 months, about 9 months, about 10 months, about 11 months, about 1 year, at least about 1 day, at least about 1 week, at least about 1 month, at least about 6 months, or at least about 1 year.

In some embodiments, the devices can be used once and then discarded; in other words, the devices can be disposable. In other embodiments, the devices can be reusable allowing a single device to sterilize at least about 10, at least about 50, at least about 100, at least about 250, at least about 500, or at least about 1,000 subgingival area, periodontal pocket(s) or area(s) of gum tissue using a single battery. In some embodiments, devices can be discarded after battery depletion. In other embodiments, a battery can be replaced and the device can be used again. In still other embodiments, the battery can be re-charged and the device used again and again.

The devices can be washable and sterilizable using conventional sterilization techniques. In some embodiments, the devices are sealed sufficiently to allow multiple washings with a detergent or alcohol based cleaner without damaging the device. Further, the devices can be sterilized using gamma irradiation techniques.

In some embodiments, the devices can automatically shut off when the battery is below the threshold to produce enough energy to sterilize the intended surface. This automatic shut off can prevent a device from not completing sterilization.

In some embodiments, a battery can be removed as needed to clean and/or sterilize the device. In other embodiments, a battery can be removed to be replaced. In some embodiments, a battery lasts for the life of the product without replacement.

An optional printed circuit board can include a processor that can execute instructions stored in memory. Instructions can include sterilization times, light intensities, indicator light scenarios, sensor inputs/outputs, and the like.

In other embodiments, the devices described herein can have independent drivers that do not require a processor. In some embodiments, the independent drivers are LED drivers. The LED drivers can use transistors which do not include using a microprocessor.

In some embodiments, the body can include a light source. In some embodiments, the body can include at least one light source, at least two light sources, at least three light sources or more.

The light source can be an incandescent bulb, a halogen bulb, a xenon bulb, a laser, a laser emitting diode, a monochromatic light, or a light emitting diode (LED). In other embodiments, the light source can be anything that will emit ultraviolet light, ultraviolet C light, or a combination thereof.

In some embodiments, the light source can deliver or project light to sterilize a subgingival area, periodontal pocket(s) or gum tissue. In some embodiments, the light is ultraviolet light. In some embodiments, the light is ultraviolet C (UVC) light. The light can be delivered from about a 3.5 mm×3.5 mm LED. In other embodiments, the LED can be about 0.5 mm×0.5 mm, about 0.5 mm×1.0 mm, about 1.0 mm×1.0 mm, about 1.5 mm×2.5 mm, about 2.0 mm×2.0 mm, about 2.5 mm×3.5 mm, about 3.0 mm×3.0 mm, about 3.5 mm×4.5 mm, about 4.0 mm×4.0 mm, about 4.5 mm×5.5 mm, or about 5.0 mm×5.0 mm.

In some embodiments, the LED driver can operate at a constant current over the device as the battery degrades. The optical power will degrade, but will remain the same.

In other, embodiments there can be LED modulation. LED modulation can be 10 rbw, 20 rbw, 30 rbw, 40 rbw, 50 rbw, at least 10 rbw, at least 20 rbw, at least 30 rbw, at least 40 rbw, at least 50 rbw, or between 15 rbw and 35 rbw. In some embodiments, there is no LED modulation but rather the devices described herein can operate using constant current mode. In other embodiments, whether the device is using constant power, constant current, or LED modulation, it can be dependent on the life of the battery. In some embodiments, the LED drivers can operate at constant current, constant power, and/or LED modulation.

In some embodiments, UVC light can be delivered with a wavelength of about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225, nm, about 230 nm, about 235 nm, about 240 nm, about 245 nm, about 250 nm, about 255 nm, about 260 nm, about 265 nm, about 270 nm, about 275 nm, about 280 nm, between about 100 nm to about 160 nm, between about 160 nm to about 210 nm, between about 210 nm to about 260 nm, or between about 220 nm to about 280 nm. In some embodiments, UVC light can be delivered with a wavelength of about 275 nm.

The higher the wavelength utilized, the less energy that is expelled allowing for the devices to be more efficient.

In other embodiments, UVB light can be delivered with a wavelength of about 280 nm, about 285 nm, about 290 nm, about 295 nm, about 300 nm, about 305 nm, about 310 nm, about 315 nm, between about 280 nm to about 300 nm, or between about 285 nm to about 315 nm.

In some embodiments, UV light, B or C, can be delivered for less than about 5 seconds or less than about 10 seconds. In some embodiments, light is delivered for between about 5 seconds and about 10 seconds. In other embodiments, UV light, B or C, can be delivered within about 1 sec, about 2 sec, about 3 sec, about 4 sec, about 5 sec, about 6 sec, about 7 sec, about 8 sec, about 9 sec, about 10 sec, about 11 sec, about 12 sec, about 13 sec, about 14 sec, about 15 sec, about 16 sec, about 17 sec, about 18 sec, about 19 sec, about 20 sec, about 21 sec, about 22 sec, about 23 sec, about 24 sec, about 25 sec, about 26 sec, about 27 sec, about 28 sec, about 29 sec, about 30 sec, about 31 sec, about 31 sec, about 32 sec, about 33 sec, about 34 sec, about 35 sec, about 36 sec, about 37 sec, about 38 sec, about 39 sec, about 40 sec, about 41 sec, about 42 sec, about 43 sec, about 44 sec, about 45 sec, about 46 sec, about 47 sec, about 48 sec, about 49 sec, about 50 sec, about 51 sec, about 52 sec, about 53 sec, about 54 sec, about 55 sec, about 56 sec, about 57 sec, about 58 sec, about 59 sec, about 60 sec, or about one minute.

In some embodiments, sterilization can be less than 10 minutes, less than 5 minutes, or less than one minute.

In some embodiments, UV light, B or C, can be delivered for less than about 1 sec, less than about 2 sec, less than about 3 sec, less than about 4 sec, less than about 5 sec, less than about 6 sec, less than about 7 sec, less than about 8 sec, less than about 9 sec, less than about 10 sec, less than about 11 sec, less than about 12 sec, less than about 13 sec, less than about 14 sec, less than about 15 sec, less than about 16 sec, less than about 17 sec, less than about 18 sec, less than about 19 sec, less than about 20 sec, less than about 21 sec, less than about 22 sec, less than about 23 sec, less than about 24 sec, less than about 25 sec, less than about 26 sec, less than about 27 sec, less than about 28 sec, less than about 29 sec, less than about 30 sec, less than about 31 sec, less than about 32 sec, less than about 33 sec, less than about 34 sec, less than bout 35 sec, less than about 36 sec, less than about 37 sec, less than about 38 sec, less than about 39 sec, less than about 40 sec, less than about 41 sec, less than about 42 sec, less than about 43 sec, less than about 44 sec, less than about 45 sec, less than about 46 sec, less than about 47 sec, less than about 48 sec, less about 49 sec, less than about 50 sec, less than about 51 sec, less than about 52 sec, less than about 53 sec, less than about 54 sec, less than about 55 sec, less than about 56 sec, less than about 57 sec, less than about 58 sec, less than about 59 sec, less than about 60 sec, or less than about one minute.

In some embodiments, UV light, B or C, can be delivered for more than about 1 sec, more than about 2 sec, more than about 3 sec, more than about 4 sec, more than about 5 sec, more than about 6 sec, more than about 7 sec, more than about 8 sec, more than about 9 sec, more than about 10 sec, more than about 11 sec, more than about 12 sec, more than about 13 sec, more than about 14 sec, more than about 15 sec, more than about 16 sec, more than about 17 sec, more than about 18 sec, more than about 19 sec, more than about 20 sec, more than about 21 sec, more than about 22 sec, more than about 23 sec, more than about 24 sec, more than about 25 sec, more than about 26 sec, more than about 27 sec, more than about 28 sec, more than about 29 sec, more than about 30 sec, more than about 31 sec, more than about 32 sec, more than about 33 sec, more than about 34 sec, more than about 35 sec, more than about 36 sec, more than about 37 sec, more than about 38 sec, more than about 39 sec, more than about 40 sec, more than about 41 sec, more than about 42 sec, more than about 43 sec, more than about 44 sec, more than about 45 sec, more than about 46 sec, more than about 47 sec, more than about 48 sec, more than about 49 sec, more than about 50 sec, more than about 51 sec, more than about 52 sec, more than about 53 sec, more than about 54 sec, more than about 55 sec, more than about 56 sec, more than about 57 sec, more than about 58 sec, more than about 59 sec, more than about 60 sec, more than about one minute, between about 0.1 sec to about 5 sec, between about 2 sec to about 3 sec, between about 5 sec to about 7 sec, between about 5 sec to about 10 sec, between about 5 sec to about 11 sec, between about 6 sec to about 12 sec, between about 11 sec to about 15 sec, between about 13 sec to about 25 sec, between about 15 sec to about 30 sec, between about 25 sec to about 35 sec, between about 25 sec to about 45 sec, or between about 25 sec to about 60 sec.

In accordance with signal processing, in some embodiments the pulsed light applied can be a square signal, a rectangular signal, a cosine squared signal, a Dirac signal, a sinc signal, a Gaussian signal, or a combination thereof.

In some embodiments, the device can deliver ultraviolet C (UVC) light to sterilize a subgingival area, periodontal pocket(s) or gum tissue. In one embodiment, the UVC light can be delivered from a light source having a size of about 3.5 mm×3.5 mm with a wavelength of about 275 nm for an interval of about 5 sec to about 10 sec at an optical power of about 10 mW to about 50 mW. In another embodiment, the UVC light can be delivered from a light source having a size of about 3.5 mm×3.5 mm with a wavelength of about 275 nm for an interval of less than about 5 sec at an optical power of about 10 mW to about 50 mW. In another embodiment, the UVC light can be delivered from a light source having a size of about 3.5 mm×3.5 mm with a wavelength of about 275 nm for an interval of less than about 10 sec at an optical power of about 10 mW to about 50 mW.

The described light delivery can effectively denature any microbe or cellular structure on the surface, space or area of a subgingival area, periodontal pocket(s), or gum tissue.

In some embodiments the optical power can be about 1 mW, about 2 mW, about 3 mW, about 4 mW, about 5 mW, about 6 mW, about 7 mW, about 8 mW, about 9 mW, about 10 mW, about 11 mW, about 12 mW, about 13 mW, about 14 mW, about 15 mW, about 16 mW, about 17 mW, about 18 mW, about 19 mW, about 20 mw, about 25 mW, about 30 mw, between about 1 mW to about 5 mW, between about 1 mW to about 10 mW, between about 5 mW to about 10 mW, between about 5 mW to about 15 mW, between about 10 mW to about 15 mW, between about 10 mW to about 20 mW, or between about 10 mW to about 30 mW. In some embodiments, the optical power can be about 10 mW. In other embodiments, the optical power can be at least 6 mW.

One or more lens is used in conjunction with a light source to focus the light onto adapter port. The lenses can be made of plastics or glass or may be formed of a transparent polymer used to make the housing. The lenses can be made to provide a particular focal length to the light. In some embodiments, curvature of the inner or outer surface of the lens, thickness of the lens, refractive index of the lens and the like can be used to provide a particular focal length. In some embodiments, focal length is generally from the light source to the adapter port. In other embodiments, the one or more lens can be a conical lens. The conical lens can be a lens with a surface that is a cone instead of the usual sphere. In some embodiments, the conical lens can be used to transform collimated light into a ring to create an approximation of a Bessel beam. In some embodiments, the one or more lens can be a conical TOPAS® lens. In other embodiments, the conical lens can reduce the time frame to focus promoting time efficiency.

In some embodiments, the light source can be at a focal length or distance from the fiber optic adapter cap. In some embodiments, the distance can be about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, less than about 1 mm, less than about 2 mm, less than about 3 mm, less than about 4 mm, less than about 5 mm, less than about 6 mm, less than about 7 mm, less than about 8 mm, less than about 9 mm, less than about 10 mm, less than about 11 mm, less than about 12 mm, less than about 13 mm, less than about 14 mm, less than about 15 mm, less than about 16 mm, less than about 17 mm, less than about 18 mm, less than about 19 mm, less than about 20 mm, more than about 1 mm, more than about 2 mm, more than about 3 mm, more than about 4 mm, more than about 5 mm, more than about 6 mm, more than about 7 mm, more than about 8 mm, more than about 9 mm, a more than bout 10 mm, more than about 11 mm, more than about 12 mm, more than about 13 mm, more than about 14 mm, more than about 15 mm, more than about 16 mm, more than about 17 mm, more than about 18 mm, more than about 19 mm, more than about 20 mm, between about 1 mm about 5 mm, between about 5 mm and about 10 mm, between about 7 mm and about 10 mm, between about 5 mm and about 15 mm, between about 8 mm and about 15 mm, or between about 14 mm and about 20 mm. The distance can be between about 5 mm to about 10 mm from the intended surface.

In some embodiments, the light output homogeneity can be characterized such that all areas of the intended surface to be sterilized receive enough exposure. In other embodiments, the least amount of light output can result in an acceptable amount of radiation to sterilize the intended surface. The optical power of the light output homogeneity can be about 6 mW, about 7 mW, about 8 mW, about 9 mW, about 10 mW, at least about 6 mW, at least about 7 mW, at least about 8 mW, at least about 9 mW, at least about 10 mW, more than about 6 mW, more than about 7 mW, more about 8 mW, more than about 9 mW, more than about 10 mW, less than about 6 mW, less than about 7 mW, less than about 8 mW, less than about 9 mW, less than about 10 mW, between about 6 mW to about 10 mW, between about 7 mW to about 9 mW, between about 7 mW to about 8 mW, or about 8 mW to about 9 mW.

In some embodiments, the devices are handheld. In some embodiments, the devices are ergonomic. The devices can be disposable and used for a single use. In other embodiments, the devices can be used multiple times.

In other embodiments, the devices described herein can include an inter-locking circuit. The inter-locking circuit can be a safety feature which can prevent the UV light from being emitted prematurely. In some embodiments, the inter-locking circuit can be an inter-locking circuit cap which is connected to the device. When the inter-locking circuit cap is connected to the device, the safety aspect prevents the UV light from being emitted prematurely.

In other embodiments, the inter-locking circuit can be connected to the fiber optic adaptor cap or to a luer to turn on the safety feature. In other embodiments, the inter-locking circuit can have an RFID chip or an optical senor configured to allow the inter-locking circuit cap and device to communicate. In some embodiments, the inter-locking safety feature can be triggered when the inter-locking circuit cap can be connected to the device and the inter-locking circuit cap and RFID/optical sensor can communicate. In some embodiments, the fiber optic adaptor cap and device can communicate via physical, mechanical, or optical communication.

Methods are also described for using the herein described devices. A user attaches the fiber optic adapter cap to a device. Fiber optic adapter cap can be probed around gum tissue and below the tissue line subgingivally. The user starts the device by activating a switch. A light indicates that the device is sterilizing the gum tissue and then changes color when the cycle is complete.

In some embodiments, the device can be prepackaged so there is no gap in exposure time. For example, the device and fiber optic adaptor cap can be prepackaged for a single use. In other embodiments, the device can be prepackaged and the fiber optic adapter cap can be separately packaged. The fiber optic adaptor cap can be disposable and for a single use.

In some embodiments, kits including the herein described devices can be provided. In one embodiment, a kit can include a device such as a device described herein in an appropriate packaging and instructions for use.

In one embodiment, a kit can include a disposable device such as a device described herein in an appropriate packaging and instructions for use.

In one embodiment, a kit can include a device such as a device described herein in an appropriate packaging, a recharging cradle, and instructions for use

### Example 1

A patient visits their dental office for a routine dental exam. Upon examination a dental hygienist determines the patient has periodontal inflammation, with redness and minor bleeding of the gum tissue. The hygienist uses a manual probe to identify periodontal pocket depth. The patient also has a history of poor dental hygiene and does not routinely brush their teeth. Tooth decay and gingivitis is also identified in one or more areas where calculus forms a tough outer layer around the tooth. The dental hygienist informs the dentist of the need to treat the aggressive microbial contamination affecting the patients subgingival. In addition to scaling, and root planning, a deep cleaning is the best course of treatment. The dentist determines the deep cleaning will involve microbial UV irradiation to prevent further tooth decay and eliminate microbial colonization.

The dental hygienist or dentist identifies the best location for UV treatment and then selects the appropriately sized and shaped disposable fiber optic adapter cap. The disposable fiber optic adapter cap is then attached to the UV device and a luer secures the optical probe into place. The fiber optic tip may or may not have specific shape, angle, or diameter. The optical probe is then slowly placed into the periodontal pocket(s) and a switch is activated by the practitioner to activate the UV device. The UV device delivers a specific timed dose of UVC light which can be 1-10 seconds. The UV light actively eliminates all the subgingival bacteria and/or microbial biofilm as a means to improving the patients gum tissue.

Multiple treatments may be needed in the same day, or within the same or subsequent weeks. Multiple treatments and multiple office visits for UV deep cleaning may be needed to return the patients flora to a neutral state promoting good health and proper dental hygiene.

### Example 2

Flexible and rigid endoscopes are regularly utilized for procedures where working instruments are passed through a lumen or multiple lumens to deliver treatment to a specific part of the patient. In the endoscopy setting, long fiber optic endoscopes are used for colonoscopy procedures where physicians rely on camera visualization and working instruments like graspers to remove polyps or abnormal growths that line the internal surface of the patient's intestines or colon. Bodily fluid or debris can routinely get lodged within the working channels as fluid and suction function act in unison to flush and clear the area of treatment. If not properly decontaminated disinfected microbial colonization can occur as a result of biofilm development. If not properly sterilized, microbial colonization and a build up of harmful bacteria can be transferred from patient to patient causing infection and serious illness or death.

In addition to routine decontamination, local site-specific UV irradiation can help sterilize the endoscope in its entirety thereby preventing the buildup of harmful biofilm that contributes to microbial contamination.

The long or short fiber optic extension is connected via luer connection to the UV device. Once secure the fiber optic extension cable, which has a spherical lens at the tip, is inserted into one of the working channels. The device is activated via manual button and the UV irradiation is delivered directly into the lumen of the endoscope. The spherical lens at the tip of the fiber optic cable permits a uniform 360 degree UV dose. The technician sterilizing the scope slowly pulls the fiber optic cable back up through the working channel. This process may be manual or it may be automatic whereby a device controls the rate sterilization and time of UV dosing, directing the fiber optic extension through the working channel in an automated format.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A sterilization device comprising:
a body comprising at least one indicator light, a battery, and a light source; and
an adapter cap;
wherein the adapter cap includes a fiber optic cable or filament.

2. The sterilization device of claim 1, wherein the battery is at least 6 volts.

3. The sterilization device of claim 1, wherein the indicator lights turn green upon sterilization.

4. The sterilization device of claim 1, wherein the light source is a light emitting diode.

5. The sterilization device of claim 1, wherein the light source emits ultraviolet C light.

6. The sterilization device of claim 1, wherein the light source has a wavelength of about 275 nm.

7. (canceled)

8. The sterilization device of claim 1, wherein the adapter cap is sterile.

9. The sterilization device of claim 1, wherein the adapter cap is disposable.

10. The sterilization device of claim 1, wherein the adapter cap is a luer connection.

11. The sterilization device of claim 1, further including a switch, at least one or more sensors, or a multi-sensor to activate the sterilization device.

12. The sterilization device of claim 1, wherein sterilization occurs in less than about 10 sec.

13. (canceled)

14. The sterilization device of claim 1, wherein microbial reduction is a 4 log microbial reduction.

15. A method of sterilization, the method comprising:
inserting a sterilization device into a mouth of a subject to apply sterilizing light to the space in the mouth,
wherein the device sterilizes the space in the mouth.

16. (canceled)

17. (canceled)

18. The method of claim 15, wherein the space in the mouth is a subgingival area, a periodontal pocket, or an area of gum tissue.

19. The method of claim 15, wherein the sterilizing light is ultraviolet C light.

20. (canceled)

21. The method of claim 19, wherein the sterilizing light has a wavelength of about 275 nm.

22. The method of claim 15, wherein the sterilization device comprises at least one indicator light, a battery, a light source, and an adapter cap.

23. The method of claim 15, wherein the light source is about 3.5 mm×3.5 mm light emitting diode.

24. (canceled)

25. The method of claim 15, wherein the sterilizing light is emitted for about 5 seconds to about 10 seconds.

26. The method of claim 22, wherein the indicator light turns green upon the completion of the sterilization a subgingival area, a periodontal pocket, or an area of gum tissue.
